(19) United States
(12) Patent Application Publication
(10) Pub. No.: US 2025/0289773 A1
(43) Pub. Date: Sep. 18, 2025

(54) Producing Ethanol from Cryogenic Separation of Syngas

(71) Applicants: Saudi Arabian Oil Company, Dhahran (SA); Korea Advanced Institute of Science and Technology (KAIST), Daejeon (KR)

(72) Inventors: Hasan Imran, Dhahran (SA); Yasmeen Aldawsari, Dhahran (SA); Ali Shakir Al Hunaidy, Qatif (SA); Kosan Roh, Daejeon (KR); Jay H. Lee, Daejeon (KR); Wonsuk Chung, Daejeon (KR)

(21) Appl. No.: 18/604,734

(22) Filed: Mar. 14, 2024

(51) Int. Cl. C07C 29/32; B01D 53/00; C01B 3/34

(52) U.S. Cl. CPC C07C 29/32; B01D 53/002; C01B 3/34; C01B 2203/0211; C01B 2203/061; C01B 2203/1241

(57) ABSTRACT

A system and method for producing ethanol, including dry reforming of methane with carbon dioxide to produce syngas, cryogenically separating carbon monoxide from syngas giving a first stream including primarily carbon monoxide and hydrogen. The method includes synthesizing methanol from the second stream via hydrogenation of carbon monoxide in the second stream and finally synthesizing ethanol via methanol homologation including the first stream of cryogenically separated carbon monoxide and a hydrogen stream.

### TECHNICAL FIELD

This disclosure relates to methods of reducing CO₂ emissions in the production of ethanol, the process comprising dry reforming of methane with CO₂ resulting in the production of syngas; cryogenic separation of CO from the syngas; methanol synthesis; and finally, methanol homologation using the cryogenically separated CO stream and hydrogen to produce ethanol.

### BACKGROUND

Due to increasing concerns about climate change, carbon capture, utilization (including conversion) and storage (CCUS) has received significant attention around the world. Among them, CO₂ conversion technologies have recently attracted much attention, as geological storage has technical challenges. The aim of CO₂ conversion is to utilize concentrated CO₂ (e.g., from CO₂ capture) as a feedstock to produce valuable chemicals via various conversion processes.

Dry reforming of methane (DRM) is one of the promising CO₂ conversion technologies for mitigating CO₂ emissions. It has a high CO₂ utilization ratio (CH₄:CO₂=1:1) and is expected to achieve significant CO₂ reduction. One of the feasible options to utilize the produced syngas (H₂ and CO) is the formation of ethanol via the following reaction, H₂+CO→CH₃OH—C₂H₅OH.

The above conventional pathway for ethanol production requires raw materials such as methanol and H₂ from steam methane reforming. The carbon footprint for this conventional process is 5.440 ton CO₂ eq/ton ethanol (EtOH)

However, 95% of the world's ethanol production is bio-based. The emissions associated with the production of ethanol from bio-sources vary drastically. It depends on the feedstock and land usage. Synthetic processes account for a small share of the ethanol production market. The two main commercial synthetic ethanol production pathways are ethylene hydration and acetic acid hydrogenation. Ethylene hydration is recommended for companies that have access to low-cost ethylene sources. However, the latter is currently getting more attention in China.

### SUMMARY

An aspect described herein provides a method of producing ethanol, including dry reforming methane (CH₄) with carbon dioxide (CO₂) to produce synthesis gas (syngas) including hydrogen (H₂), carbon monoxide (CO), and CO₂. This is followed by cryogenically separating CO from the syngas and supplying it as a first stream primarily including CO and a second stream including H₂, CO, CO₂. The method includes synthesizing methanol (CH₃OH) from the second stream via the hydrogenation of CO in the second stream, synthesizing ethanol from the methanol, CO from the first stream, and renewable hydrogen, via methanol homologation.

Another aspect relates to a system for producing ethanol, including converting methane (CH₄) and carbon dioxide (CO₂) in a dry reformer vessel into hydrogen (H₂) and carbon monoxide (CO), discharging syngas from the dry reformer vessel via a discharge conduit to a cryogenic separation system including a cryogenic distillation column, wherein the syngas includes H₂, CO, and CO₂. The method includes cryogenically separating CO from the syngas via the cryogenic separation system and discharging from the cryogenic separation system a first stream including primarily CO to a methanol homologation system having a methanol homologation reactor vessel, which is operationally coupled to the cryogenic separation system. The system includes discharging from the dry reforming system a second stream including H₂, CO, and CO₂ via a feed conduit to a methanol synthesis system. The methanol synthesis system has a methanol synthesis reactor vessel. The system includes bypassing a portion of the syngas from the discharge conduit around the cryogenic separation system to the feed conduit, and synthesizing methanol (CH₃OH) from the second stream in the methanol synthesis system via hydrogenation of CO from the second stream in the methanol synthesis reactor vessel.

The details of one or more implementations are set forth in the accompanying drawings and the description below. Other features and advantages will become apparent from the description, drawings, and claims.

### BRIEF DESCRIPTION OF DRAWINGS

FIG. 1 is a drawing of a system that produces ethanol by the dry reforming of methane and CO₂.

FIG. 2 is a block flow diagram of a method of producing ethanol.

FIG. 3 is a process simulation diagram representing the dry reforming of methane pathway including the cryogenic separation system and methanol synthesis system.

FIG. 4 is a process simulation diagram representing the methanol homologation system to produce ethanol.

FIG. 5 is a process simulation diagram representing the ethanol separation.

### DETAILED DESCRIPTION

Aspects described herein provide methods and systems for the utilization of greenhouse gases such as carbon dioxide to produce a useful chemical like ethanol. This disclosure includes the utilization of concentrated CO₂ (e.g., from CO₂ capture) as a feedstock to produce valuable chemicals via various conversion processes.

In implementations herein, dry reforming of CH₄ with CO₂ produces 2 moles of hydrogen and 1 mole of CO from 1 mole of CO₂ and 1 mole of CH₄. This is a CO₂ conversion technology for mitigating CO₂ emissions. The dry reforming of methane (DRM) has relatively high CO₂ utilization ratio (e.g., molar ratio CH₄:CO₂=1:1) and thus facilitates CO₂ reduction. Implementations herein utilize CO₂ as a feedstock via DRM to produce ethanol (C₂H₅OH) via methanol homologation.

This disclosure relates to the processing of CO₂ to produce synthesis gas (syngas) by dry reforming of methane and the cryogenic separation of syngas. The syngas produced from DRM may be primarily hydrogen (H₂) and carbon monoxide (CO) at a molar ratio of 1:1 based on the ideal thermodynamic equilibrium. The syngas produced from DRM may also include CO₂. The ethanol produced via syngas may proceed via the overall reaction H₂+CO→CH₃OH—C₂H₅OH. This method is different than the conventional production of ethanol which is synthesized by the fermentation of sugars and/or cellulosic material. Further, the implementation herein relies on sources such as CO and H₂ from DRM and not from steam methane reforming (SMR). Methane is obtained from a rich source such as natural gas. The syngas produced from DRM process is cryogenically separated to produce a first stream that primarily includes CO.

A second stream from the cryogenic separator includes CO, CO₂, and H₂ that are fed into a methanol synthesis system. Additionally, a bypass stream which includes syngas from the dry reforming system, is discharged via a bypass around the cryogenic separation system into the methanol synthesis system. The generated methanol undergoes homologation along with the first CO stream and hydrogen to produce ethanol. A number of processes are integrated, including the DRM to provide manufacturing materials and ethanol synthesis by methanol formation and methanol homologation. Finally, other impurities are separated. In support, models were created to assess the associated CO₂ emission in comparison with the conventional ethanol production pathway.

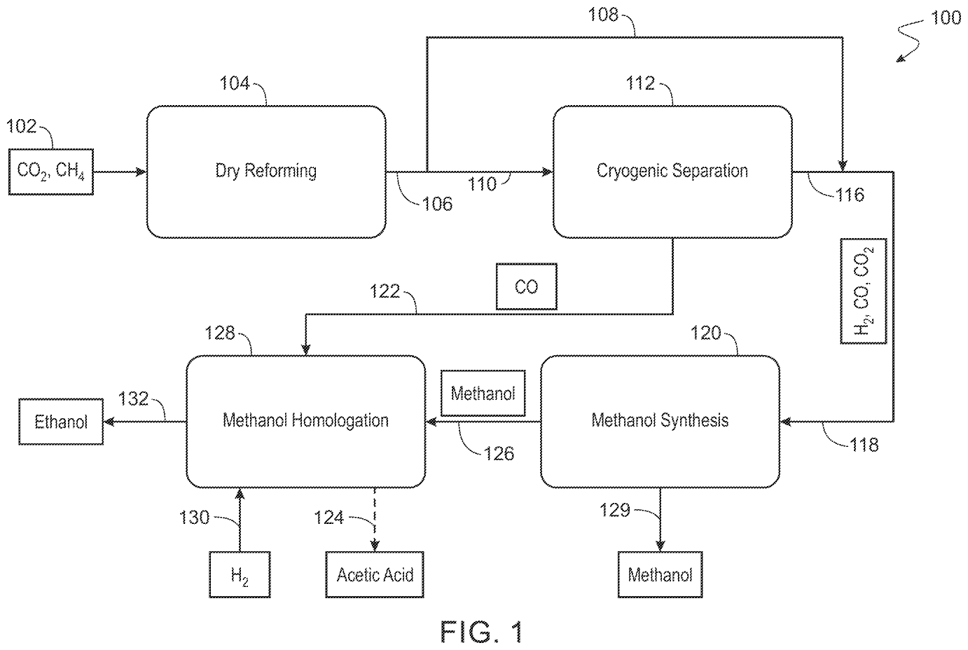

FIG. 1 is a drawing of a system 100 that synthesizes ethanol. The feed 102 utilizes CO₂ and CH₄. Utilization of CO₂ as a feed has an advantage of CO₂ emissions reduction at a facility. In implementations, the CH₄ may be fed in as a methane-rich stream, such as natural gas.

A dry reforming system 104 receives the feed 102 and proceeds via reaction CO₂+CH₄→2H₂+2CO. The dry reforming system 104 includes a dry reformer vessel which may contain a catalyst. The dry reforming vessel may be, for instance, a fixed-bed reactor or a fluidized bed reactor having the catalyst. The reforming catalyst may include, for example, noble metals such as nickel (Ni), or Ni alloys. In some implementations, the catalyst is magnesium oxide (MgO) or MgO nanoparticles. The MgO or MgO nanoparticles may be promoted with Ni and/or molybdenum (Mo), for example. Other reforming catalysts are applicable. The dry reforming reaction via the catalyst gives syngas comprising H₂ and CO. The dry reforming reaction may be represented by CH₄+CO₂→2H₂+2CO. The molar ratio of H₂ to CO in the syngas discharge 106 based on the ideal thermodynamic equilibrium is 1:1, but in practice it can be different than 1:1. Unreacted CH₄ may discharge in the syngas discharge 106 stream. In some implementations, unreacted CH₄ may be separated from the discharged syngas 106 and recycled to the dry reformer vessel. The syngas discharge 106 may also include CO₂, for example, at less than 10 mole percent (mol %).

In operation, the dry reforming system 104 converts CO₂ and CH₄ into syngas. A portion of the syngas discharge 106 is fed as an inlet stream of syngas 110 into the cryogenic separation system. The example operating conditions of the dry reformer system 104 are 2 bar to 28 bar and temperature range 500° C. to 1100° C., as used herein, bar is in absolute pressure. The operating pressure in the dry reforming system 104 may be, for example, in the range of 2 to 28 bar, or less than 30 bar. In some implementations, the operating pressure may exceed 30 bar to provide additional motive force for flow of the discharged syngas 106 to downstream processing. The operating temperature of the dry reforming system 104 may be, for example, in the ranges of 500° C. to 1100° C., at least 500° C., less than 1000° C. The dry reforming reaction may generally be endothermic. The dry reforming system 104 may have a jacket for heat transfer and temperature control. In operation, a heat transfer fluid (heating medium) may flow through the jacket for temperature control. In other implementations, electrical heaters may provide heat for the endothermic dry reforming reaction. The electrical heaters may be placed in the dry reforming vessel or on the external surface of dry reforming vessel. In yet other implementations, the dry reformer vessel may be placed in a furnace (e.g., a direct fired heater) to receive heat and for temperature control. Other configurations of heat transfer and temperature control of the dry reforming system 104 are applicable.

The dry reforming system 104 includes feed conduit(s) for the feed 102. The CO₂ and CH₄ (or natural gas) may be introduced as a combined stream or in separate streams to the dry reforming system 104. In implementations, a feed conduit may add CO₂ to a feed conduit conveying CH₄ (or natural gas) for introduction to the dry reformer vessel. The flow rate (e.g., volumetric rate, mass rate, or molar rate) may be controlled using flow control valve(s) that may be disposed along the respective supply lines, or by a mechanical compressor, or a combination thereof. The ratio (e.g., molar, volumetric, or mass) of the CH₄ or natural gas to the CO₂ may be modulated (via one or more of the control valves) by controlling the flow rate of at least one of the flow rates of the CH₄ (or natural gas) or CO₂. The dry reformer vessel has an inlet to receive the feed 102. The inlet may be, for example, a nozzle having a flange or threaded connection for coupling to a feed conduit conveying the feed 102.

The dry reformer system 104 includes a discharge conduit, or dry reformer discharge conduit, to carry the syngas 106. The dry reformer vessel may have an outlet, (e.g., a nozzle with a flanged or screwed connection, for the discharge of the produced syngas 106 through the discharge conduit for downstream processing. The discharge conduit may extend to the cryogenic separation system 112. The discharge conduit may be labeled as a conduit coupled to a cryogenic separation feed conduit.

In implementations, steam methane reforming is not employed. Instead, dry reforming of methane as discussed above is employed to produce syngas 106 that has a beneficial CH₄ to CO₂ ratio of 1. After processing of the discharged syngas 110 via the downstream cryogenic separation system 112, the CO 122 from the syngas 110 is removed for supply to the methanol homologation system 128. The remaining syngas feed 116 (processed syngas) discharged from the cryogenic separation system may have a desired or adjustable range for the molar ratio of H₂ to CO for methanol synthesis.

The syngas 110 may be pretreated to remove impurities, such as water, which would freeze at cryogenic temperatures. In implementations, the cryogenic separation system 112 may discharge at least two streams including a first and second stream. The first stream may be the separated CO 122 that may be discharged to the methanol homologation system 128. The second stream may be the feed 116 (including the processed syngas) discharged to the methanol synthesis system 120.

The cryogenic separation system 112 may include a refrigeration system to provide temperatures in the range of −150° C. to −120° C. The cryogenic separation system 112 includes a mechanical gas compressor, heat exchangers, and separation columns including a distillation column. The CO 122 stream can be separated from syngas 110 by cooling it until liquified and then distilling at component boiling temperatures. The separation columns may include a wash column, stripping column, and the like.

In addition to the cryogenic distillation, unit operations may include a methane wash, partial condensation, or CO wash, among others. The operation can be tailored to meet a desired percent purity of the CO 122. In some implementations, the cryogenic separation system 112 may be referred to as a cold box utilized for separation of CO from syngas. As mentioned, the CO 122 separated from the syngas 110 may be fed to the methanol homologation system 128 that discharges ethanol (C₂H₅OH) 132. The operation of the methanol homologation system 128 is discussed below.

In implementations, the amount of CO 122 removed from the syngas 110 may approximately double the molar ratio of H₂ to CO in the remaining syngas (processed syngas) 116 that discharges as the second stream. In examples, the molar ratio of H₂ to CO in the syngas discharged from the dry reforming system 104 may be about 1 (2 moles H₂ per 2 moles CO), and the molar ratio of H₂ to CO in the processed syngas 116 discharged from the cryogenic distillation column may be about 2 (2 moles H₂ per 1 mole CO).

A bypass feed, including a bypass conduit 108, around the cryogenic separation system 112 may introduce some of the syngas discharge 106 into the processed syngas feed 116 that discharges out of the cryogenic separation system 112. The reason to introduce the bypass conduit 108 into the processed syngas discharge feed 116 is to adjust the molar composition of the processed syngas discharged from the cryogenic separation system 112 or to adjust the molar relationship among the H₂, CO₂, and CO inlet feed 118 gases which may benefit the methanol synthesis reaction in the methanol synthesis system 120.

In implementations, the bypass conduit 108 may route the bypass portion of the syngas 106 from the discharge conduit of the dry reformer system 104 to the conduit conveying the feed 116 downstream of the cryogenic separation system 112. This conduit conveying the feed 116 may be labeled as a discharge conduit from the cryogenic separation system or as a feed conduit (methanol-synthesis feed conduit) to the methanol synthesis system 120. In some implementations, the bypass conduit 108 may include a flow control valve disposed along the conduit. The control valve may be used to control the amount of syngas that flows through the bypass conduit 108. In the depicted FIG. 1, 0.2 of the syngas 106 based on volume may bypass the cryogenic separation system 112 into the bypass conduit 108. In some instances, no syngas 106 bypasses the cryogenic separation system 112 (e.g., when the control valve is closed). The amount of syngas 106 based on volume that bypasses the cryogenic separation system 112 can be, for example, in the range of 0.05 to 0.4 of syngas 106.

Components of the feed 116 with or without the syngas from the bypass conduit 108 may generally include at least H₂, CO₂, and CO. An online instrument, for example a gas chromatograph analyzer may be disposed along the discharge conduit from the cryogenic separation system 112. The online instrument may be used to measure the feed composition of the stream. The discharge conduit from the cryogenic separation system 112 may also be coupled to the feed conduit 118 to the methanol synthesis system 120. In implementations, the molar ratio of H₂ to CO in the feed 118 may be specified and maintained at about 2 or at least 2, which may be beneficial for the downstream methanol synthesis.

In some implementations, the relationship of H₂, CO₂, and CO in the feed 118 may be controlled per the following module ratio, where is the number of moles:

$$η = \frac{{η}_{{H}_{2}} - {η}_{{CO}_{2}}}{{η}_{CO} - {η}_{{CO}_{2}}}$$

This module may be controlled, for example, to a value of 2 or at least 2, which can be beneficial for methanol synthesis. In certain implementations, the set point of the control valve along the bypass conduit 108 may be adjusted to give the desired value of the module ratio in the feed 118.

The methanol synthesis system 120 may receive the feed 118. The feed 118 has CO and H₂ and may have CO₂. The methanol synthesis system 120 may react the CO and H₂ over a catalyst (e.g., Cu-based catalyst) to produce methanol (CH₃OH) 126. This methanol synthesis can be characterized as a CO hydrogenation reaction. Methanol can be produced by the hydrogenation of CO or CO₂ over the catalyst. Catalytic conversion of H₂ and CO into methanol can be in a gas-phase reactor. The methanol synthesis reaction can proceed via reaction 2H₂+CO→CH₃OH. Thus, it is beneficial to maintain the molar ratio of 2 or above 2 in the feed 118 such that any side reactions may be suppressed. In implementations, the molar ratio of H₂ to CO being larger than 2 in the feed 118 may advance the conversion of CO₂ in the methanol synthesis reaction and facilitate to suppress side reactions in the methanol synthesis.

The methanol synthesis can be labeled as the hydrogenation of carbon oxides (COₓ) to methanol. The carbon oxides are CO and CO₂. In addition to minor amounts of other components, the feed 118 may include ternary mixtures of H₂+CO+CO₂. The ternary mixtures can have varying molar ratios of CO₂/CO and H₂/COₓ. These molar ratios may be maintained, for example, via the bypass conduit 108 and/or operation of the cryogenic separation system 112. The illustrated methanol synthesis system 120 has a methanol synthesis reactor vessel which may be a gas phase reactor. The reactor vessel has a copper-based (Cu-based) catalyst. The catalyst may include copper, zinc oxide (ZnO), alumina, magnesia, copper oxide (CuO), or aluminum oxide (Al₂O₃), or mixtures thereof. In certain implementations, the catalyst is a mixture of copper and zinc oxides, supported on alumina. For instance, the catalyst is Cu—ZnO—Al₂O₃, sometimes modified with ingredients contributing to the increase of the copper dispersion and stability. Other catalysts are applicable. The reactor vessel may be a fixed-bed reactor having the catalyst in a fixed bed.

The operating conditions of the methanol synthesis reactor vessel may include an operating pressure bar in the range of 30 bar to 120 bar. The operating temperature may be, for example in a range of 220° C. to 280° C. The operating conditions may be outside of these numerical ranges. The methanol synthesis reaction 2H₂+CO→CH₃OH is generally exothermic. Therefore, heat may be removed from the vessel using a heat transfer jacket, a recirculation heat exchanger, or other heat transfer system. In implementations, the heat removed may be used as a source of heat utilized for other operations such as steam or electricity generation.

A minor proportion of byproducts may be generated in the methanol synthesis reaction. The byproducts may include, for instance, at least one of CH₄, methyl formate, higher alcohols, or acetone. In implementations, unreacted CO, unreacted H₂, and unreacted methanol 129 discharged from the reactor vessel may be recycled to the reactor vessel or may be drawn as a byproduct if desired.

The final product stream 126 discharged from the methanol synthesis system 120 may include at least 70 vol % methanol. The methanol synthesis system 120 may discharge the stream 126 via a conduit into the methanol homologation system 128. This product stream 126, e.g., having methanol at greater than 70 volume percent (vol %) from the methanol synthesis system 120, may be the feed to the methanol homologation system 128.

The methanol homologation system 128 receives the methanol stream 126 which includes methanol with a composition of 70 vol %, CO stream 122 from the cryogenic separation system 112, and H₂ stream 130 from a renewable energy source. The H₂ may also be obtained from other sources such as electrolysis, steam methane reforming system, renewable electrolysis, and the like. In implementations herein, the H₂ may be obtained from renewable sources. The methanol homologation system includes a reactor vessel that may contain a catalyst to advance the methanol homologation process. The methanol homologation process can proceed via the reaction CH₃OH+2H₂+CO→C₂H₅OH+H₂O. The reaction may be a liquid-phase reaction. The reactor vessel may have transition metals such as ruthenium-cobalt (Ru—Co based) catalyst. The catalyst may include rutheium, cobalt, manganese, nickel, platinum, lithium, iron, or mixtures thereof. In certain implementations, the catalyst is a mixture of rutheium and cobalt with halide promoters. Other catalysts are applicable. The reactor vessel may be a fixed-bed reactor having the catalyst in a fixed bed.

The operating conditions of the methanol homologation system 128 may include a temperature range of 225-275° C. and pressure in the range of 50 to 175 bar. The operating conditions may be outside these numerical ranges. The use of the solid phase catalyst may help to suppress the formation of side reactions like the formation of aldehydes, ethers, acetic acid 124. This process leads to the synthesis of ethanol (C₂H₅OH) 132 along with water, as a product of the methanol homologation reaction. The ethanol 132 obtained may have a composition of 70 mol %. The other impurities formed from the reaction may be separated. The obtained ethanol 132 may undergo separation or purification processes. This process may include using extractive distillation, azeotropic distillation, and the like. The process can also be adapted to form acetic acid and methanol as by products, thereby generating income streams from multiple chemicals.

The ethanol production system 100 may include a control system that facilitates the operation of the system 100, such as supply or discharge of flow streams and associated control valves, control of operating temperatures and operating pressures, and control of reactors, distillation columns, heat exchangers and so on. The control system may include a processor and memory storing code (such as logic and instructions) executed by the processor to perform calculations and direct operations of the system 100. The processor may be one or more processors and each processor may have one or more cores. The processor(s) may include a microprocessor, central processing unit (CPU), graphic processing unit (GPU), controller card, circuit board, or other circuitry. The memory may include volatile memory, non-volatile memory, and firmware. The control system may include a desktop computer, laptop computer, computer server, control panels, programmable logic controller (PLC), distributed computing system (DCS), controllers, actuators, or control cards. The control system may be communicatively coupled to a remote computing system that performs calculations and provides direction. The control system may receive user input or remote computer input that specifies the set points of control devices or other control components in the ethanol production system 100. In some implementations, the control system may calculate or otherwise determine set points of control devices. Some implementations may include a control room that can be a center of activity, facilitating monitoring and control of the process or facility. The control room may contain a human machine interface (HMI), which is a computer, for example, which runs specialized software to provide a user-interface for the control system. The HMI may present the user with graphical version of the process. There may be multiple HMO consoles, with varying degrees of access to data. The control system may also employ local control (such as distributed controllers, local control panels, and the like) distributed in the system 100.

The amount of the bypass conduit 108 flow such as mass per time, volume per time, fractional amount of incoming syngas 106 may be specified and controlled such that the feed 116 has the desired or specified value for the module ratio or for the molar ratio of H₂ to CO, or both. In implementations, the set point of the bypass flow control valve may be set manually or set by the master controller in the control system based on the feedback received from the online instrument.

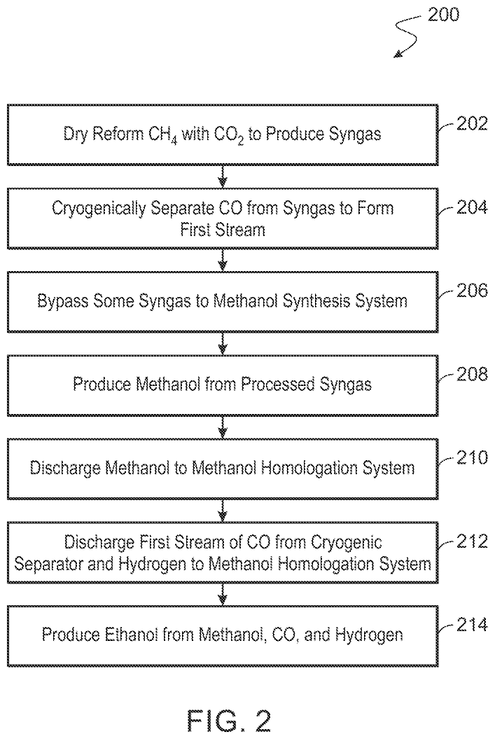

FIG. 2 is a method 200 for producing ethanol. As mentioned in the description of FIG. 1, the synthesis of ethanol occurs via the methanol homologation reaction utilizing the cryogenically separated CO stream from syngas from dry reforming. The dry reforming process produces H₂ and CO in a molar ratio of 1, thereby making the syngas beneficial for implementations by utilizing some of the CO for methanol homologation. The rest of the syngas that is not used for methanol homologation is used for methanol synthesis. The technique cryogenically separates the CO from syngas for methanol homologation. It also gives the approximate molar relationship (2H₂+1CO) for methanol production. Moreover, the uniqueness of dry reforming methane is a beneficial starting point for this approach. Lastly, as discussed below, implementations may give lower final product cost and lesser CO₂ footprint, as compared to the conventional process.

At block 202, the method herein include dry reforming CH₄ with CO₂ to produce syngas (H₂ and CO). The produced syngas may also include CO₂. The dry reforming process may include a dry reformer vessel and may proceed via the reaction CO₂+CH₄→2H₂+2CO. The reaction gives H₂ and CO which may have an approximate molar ratio of 1.

At block 204, the method includes cryogenically separating CO from the syngas and giving a first stream that comprises primarily of CO and a second stream that includes H₂, CO, and CO₂. The method may include discharging syngas from the dry reformer vessel via a discharge conduit to a cryogenic separation system. The cryogenic separation system may discharge a first stream that is primarily CO to the methanol homologation system and a second stream (processed syngas) via a feed conduit to a methanol synthesis system. In implementations, the removal of CO may approximately double the molar ratio of H₂ to CO.

At block 206, the method can include bypassing some portion of the syngas around the cryogenic separation system via a bypass conduit to the methanol synthesis system. The bypass portion of the syngas is discharged from the dry reforming system. In particular, the method may include adding the bypass portion to the feed conduit of the second stream to the methanol synthesis system. The second stream is the processed syngas (minus the portion of CO separated as first stream) coming out of the cryogenic separation system via a discharge conduit. In implementations, the bypass portion as a volume percent of the syngas discharged from the dry reforming may be, for example, in a range of 5% to 40%.

At block 208, the method includes synthesizing methanol via the hydrogenation of CO in the second stream. The method can include synthesizing methanol in a methanol synthesis reactor that may contain a catalyst. The hydrogenation of CO may involve the reaction 2H₂+CO→CH₃OH. The second stream as feed may have a molar ratio of H₂ to CO of at least 2.

At block 210, the methanol synthesis in the methanol synthesis system may be discharged via an outlet conduit to the methanol homologation system. The methanol may be purified to the desired purity before being discharged to the methanol homologation system.

At block 212, the method includes receiving the first stream primarily CO from the cryogenic separation system, methanol from the methanol synthesis system, and hydrogen from a renewable source. H₂ may be supplied from other sources as well. The methanol homologation system may include a reactor vessel that has a catalyst to advance the homologation reaction to produce ethanol. The ethanol formation may proceed via reaction CH₃OH+2H₂+CO→C₂H₅OH+H₂O.

At block 214, the method includes generating ethanol from the methanol homologation system. The impurities formed are separated from the desired product. The produced ethanol may undergo further separation via a distillation process to obtain the desired purity.

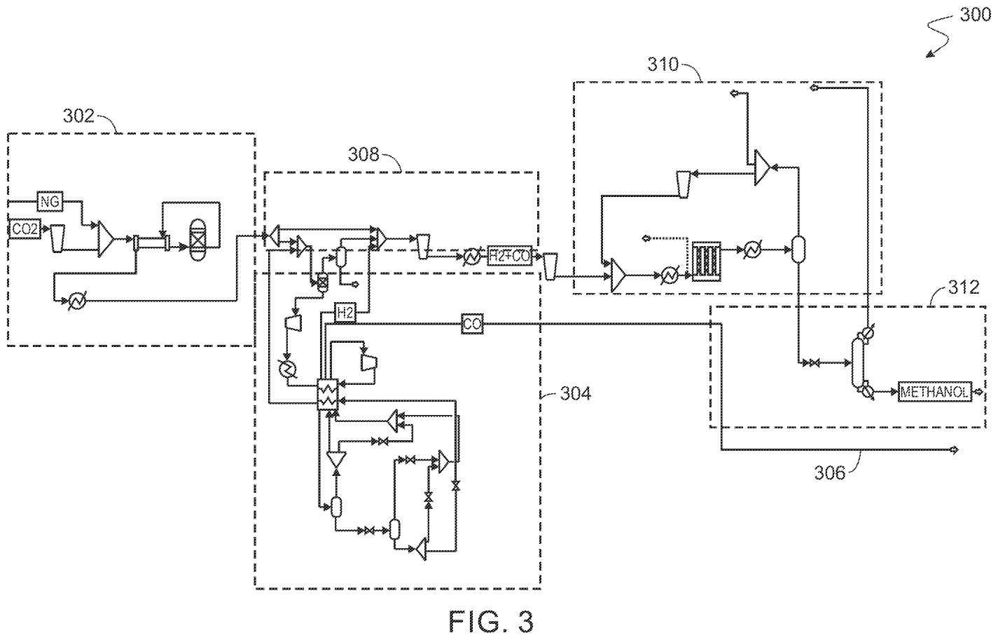

FIG. 3 is an Aspen Plus® version 8.8 process diagram 300 representing the dry reforming methane (DRM) process and the pathway to methanol synthesis including the cryogenic separation system. As mentioned in FIG. 1 and FIG. 2, the dry reforming process includes the production of syngas using CO₂ and CH₄ (usually from natural gas source) as the inlet feed.

At block 302, the process includes dry reforming of CH₄ with CO₂ as the inlet feed. The dry reforming system may include a gas compressor to feed CO₂ and CH₄ into a mixer. In some implementations the gas streams may be fed directly into a dry reforming vessel reactor. The DRM includes a reactor vessel and feed-effluent heat exchanger (FEHE). The dry reforming process is endothermic. The heat exchanger is used for temperature control and to supply heat to the reactor vessel. The dry reforming reaction may be represented by CH₄+CO₂→2H₂+2CO. The dry reforming process produces syngas that includes H₂ and CO. The molar ratio of H₂ to CO in the produced syngas based on the ideal thermodynamic equilibrium is 1, but in practice it can be different than 1:1. The processed syngas is fed via a conduit into a cryogenic separation system.

At block 304, a cryogenic separation system receives the feed from the DRM system. The cryogenic separation system includes a refrigeration system to maintain the temperature between −150° C. to −120° C. The cryogenic separation system includes a mechanical gas compressor, heat exchangers, and separation columns including a distillation column. A first stream 306 comprising primarily CO is discharged from the cryogenic separation system A second stream that comprises of CO, CO₂, H₂ is discharged via a discharge conduit from the cryogenic separation system into the methanol synthesis system.

At block 308, a bypass conduit receives a portion of the syngas from the dry reforming system that is fed into the discharge conduit of the cryogenic separation system which leads to the methanol synthesis system. The bypass conduit includes a bypass valve that can be used to regulate the flow of syngas from the dry reforming system. The bypass conduit feeds into a discharge conduit of the cryogenic separation system also labeled as methanol synthesis system feed conduit. The primary purpose of the bypass conduit is to regulate the molar ratio of H₂ and CO that feeds into the methanol synthesis system.

At block 310, the methanol synthesis reactor receives an inlet feed that includes CO, CO₂, and H₂. The methanol synthesis system includes a reactor that may have a catalyst to advance the methanol synthesis reaction. Methanol is produced by the hydrogenation of CO and proceeds via the reaction 2H₂+CO→CH₃OH.

At block 312, the generated methanol enters the methanol separation system for further separation and purification. The methanol separation system includes a distillation column, and heat transfer system, among others. among other components. Distillation column is used to separate the impurities from methanol and the separated impurities may be recycled back to the methanol reactor. The feed from the methanol separation system enters the methanol homologation unit via a discharge conduit.

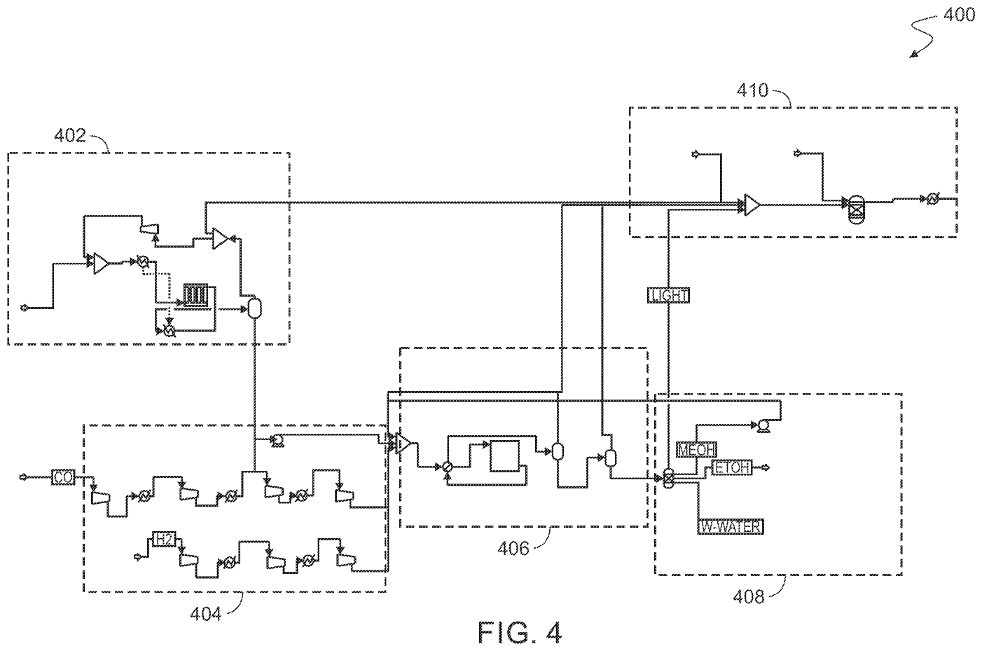

FIG. 4 is an Aspen Plus® version 8.8 process diagram 400 representing the ethanol synthesis process via the methanol homologation reaction. At block 402, the methanol synthesis system receives the feed of processed syngas from the cryogenic separation system that includes H₂, CO, and CO₂ and the syngas feed from the bypass conduit discharged from the DRM system. The molar ratio of the feed H₂ and CO is regulated by adjusting the flow rate in the bypass valve, disposed along the bypass conduit. Methanol synthesis takes place in the reactor vessel that includes catalyst and proceeds via the reaction 2H₂+CO→CH₃OH. Methanol undergoes a separation process to remove the impurities from the produced methanol.

Block 404 represents the feed gas compression which receives the first stream from the cryogenic separation system that primarily includes CO and a H₂ stream that may be obtained from a renewable source or from other sources such as steam reforming methane, electrolysis, and the like. The discharge from the feed gas compression system is fed as an inlet to the methanol homologation system.

At block 406, the methanol homologation system receives the methanol from the methanol synthesis system, the first stream from the cryogenic separation system that primarily includes CO, and the H₂ stream. The methanol homologation system includes a reactor vessel such as a fixed bed reactor or a fluidized bed reactor that may include a catalyst for the homologation reaction. The process proceeds via the reaction CH₃OH+2H₂+CO→C₂H₅OH+H₂O. The generated ethanol is separated from the water and unreacted methanol, using a distillation process.

At block 408, the generated ethanol undergoes a separation process to obtain ethanol of the desired purity. The separation process may utilize single-stage or multi-stage distillation columns. The distillation process may include extractive distillation (Gill et al, Brazil. J. Chem. Eng. 25, 207-215, 2008), which uses ethylene glycol to break the azeotrope formed in the process. The separation energy for the process consumes approximately 1.5 GJ steam/ton of ethanol produced.

At block 410, waste heat from the exothermic reaction of methanol synthesis and from other applicable unit operations in the facility, is used to generate steam at high pressure and high temperature. The generated steam may be used for multiple purposes such as for the ethanol separation process. In other implementations, the steam may be used to generate power in the steam turbine. This system may be labeled as heat recovery steam generation (HRSG) system.

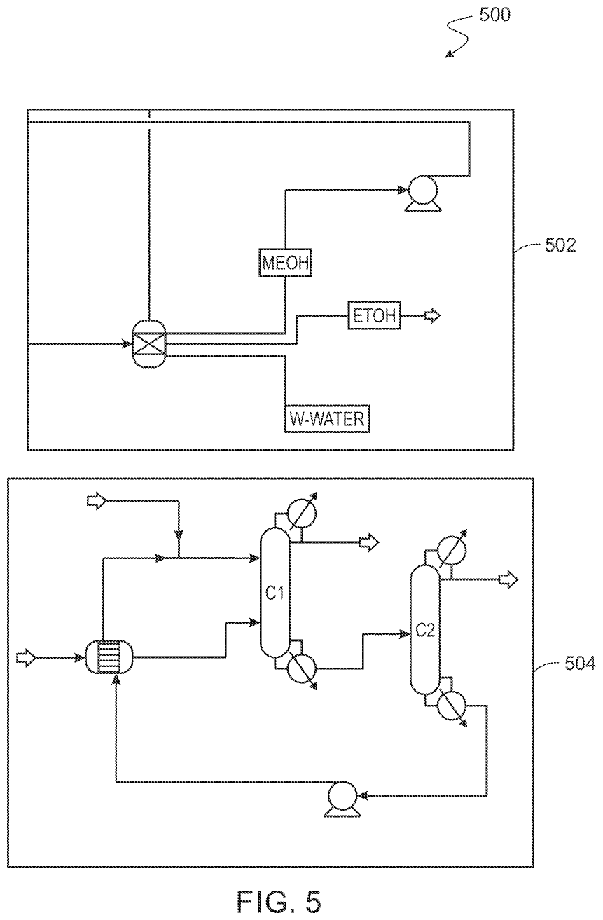

FIG. 5 is an Aspen Plus® version 8.8 process diagram 500 representing the ethanol separation process. At block 502, the separation system that includes a distillation column among other components, receives the feed ethanol generated in the methanol homologation reactor vessel, along with water and unreacted methanol. The separation process may include an extractive distillation process that uses ethylene glycol to break the azeotropic mixture to generate ethanol of the desired purity.

Block 504 represents a multi-stage separation process, which may include multiple distillation columns to separate ethanol from a mixture of ethanol, water, and unreacted methanol to generate ethanol of the desired purity. The process can be modified to produce methanol and acetic acid as by products in addition to ethanol.

The process simulator Aspen Plus® version 8.8 is utilized to model the ethanol production system 100 of FIG. 1 from the feed including CH₄ and CO₂. The system may be characterized as a DRM pathway for synthesizing ethanol via a methanol homologation pathway. A modeling study has been conducted which focuses on the methanol homologation path. To evaluate the feasibility of the process, process design and simulation were performed using the process simulator to get the energy and flows parameters. The numbers from Aspen are fed into an analysis program called ArKaTAC3, (Aramco/KAIST-Tool for Analysis of CO₂ capture & Conversion systems), to conduct CO₂ lifecycle assessment (LCA), examining the feasibility of the designed process through the analysis program by comparing it with the conventional ethanol production pathway.

Life cycle assessment (LCA) of CO₂ is a general methodology for assessing environmental impact associated with the life cycle of a commercial product or process. An example of the CO₂ LCA associated with system 100 of FIG. 1 is given in Table 1. An ethanol production process via the DRM-based pathway, shows a net CO₂ emission of 0.957 ton CO₂/ton ethanol (EtOH) produced. This number is significantly lower than the conventional ethanol production process that gives a net emission of 5.440 ton CO₂/ton EtOH. In Table 1, “in” is the amount of CO₂ uptake by the process and “out” is the amount of CO₂ emitted. The ArKaTAC3 tool is used to calculate the process parameters. In Table 1, feed CO₂ represents the amount of CO₂ fed into the dry reforming system. CO₂ capture represents the amount of CO₂ emissions released from the energy spent on the indirect carbon capture process using monoethanolamine (MEA). The steam for the CO₂ capture process is obtained from concentrated solar power (1.6 kg CO2 eq/GJ).

It is assumed that the CO₂ capture energy is 3.5 GJ/ton CO₂. This value is equivalent to coal-fired power plant. NG purchase refers to the CO₂ emissions associated with natural gas (natural gas is used as raw material) acquisition. H₂ purchase refers to the CO₂ emissions associated with H₂ manufacturing that is used as a raw material for the process in the methanol homologation reaction. Process emissions are indirect CO₂ emissions related to utilities purchase or production in the facility. Direct emissions are actual CO₂ emissions from the overall ethanol production process. The total CO₂ emission is the addition of all the “out” column numbers (2.911 ton CO₂/ton EtOH). They represent the various processes that emit CO₂ for the ethanol production system 100 in FIG. 1. The CO₂ intake by the dry reforming of methane process is 1.953 ton CO₂/ton EtOH. Therefore, the net CO₂ emission is 0.958 ton CO₂/ton EtOH (2.911-1.953=0.958). The calculated parameters are made on the assumption that a) there is a complete conversion of methane and CO₂ to syngas in the DRM and b) fuel for the process is assumed to be methane (CH₄) and not natural gas, as would be the case in the commercial process. A conventional ethanol production process emits 5.440 ton CO₂/ton EtOH produced. The current DRM based process saves 4.482 ton CO₂/ton EtOH produced.

TABLE 1
CO₂ LCA associated with the system of FIG. 1

| ton CO₂/ton EtOH | In | Out |
|---|---|---|
| Feed CO₂ | 1.953 | |
| CO2 capture | | 0.011 |
| NG purchase (extract, transport) | | 0.429 |
| H2 purchase | | 0.070 |
| Process | | 0.082 |
| Direct Emissions | | 2.319 |
| Total | 1.953 | 2.911 |

A second example for the ethanol production process via the DRM pathway using methanol homologation is studied using the ArKaTAC3 tool. In this example, hydrogen manufactured from a conventional source such as steam methane reforming is used. The LCA results are presented in Table 2. The net CO₂ emission is 2.666 ton CO₂/ton EtOH, which is still lower than the conventional ethanol production process (5.440 ton CO₂/ton EtOH).

TABLE 2
CO₂ LCA associated with the system of FIG. 1 when the
hydrogen source is from stream reforming methane

| ton CO₂/ton EtOH | In | Out |
|---|---|---|
| Feed CO₂ | 1.953 | |
| CO2 capture | | 0.661 |
| NG purchase (extract, transport) | | 0.429 |
| H2 purchase | | 1.181 |
| Process | | 0.158 |
| Direct Emissions | | 2.319 |
| Total | 1.953 | 4.619 |

Therefore, ethanol production by dry reforming CH₄ with CO₂ and methanol homologation is a feasible commercial process and leads to a reduction in the net CO₂ emissions compared to the conventional ethanol production process. In view of the example values discussed above, certain ethanol production implementations of the present technique in comparison to the conventional process of ethanol production (bio-based process) can reduce CO₂ emissions by about 82%.

Other implementations are also within the scope of the following claims.

What is claimed is:

1. A method for producing ethanol, comprising:
dry reforming of methane (CH₄) with carbon dioxide (CO₂) to produce a synthetic gas (syngas) stream comprising hydrogen, carbon monoxide (CO), and carbon dioxide;
separating carbon monoxide from the syngas cryogenically to form a first stream comprising primarily carbon monoxide and a second stream comprising carbon dioxide, carbon monoxide, and hydrogen;
forming methanol (CH₃OH) from the second stream by the hydrogenation of carbon monoxide; and
producing ethanol (C₂H₅OH) from methanol, the first cryogenically separated carbon monoxide stream and hydrogen, wherein generating ethanol comprises homologation of methanol.

2. The method of claim 1, wherein dry reforming methane comprises a reaction:
CO₂+CH₄→2H₂+2CO.

3. The method of claim 1, comprising adding a bypass portion of the syngas to the second stream, wherein the bypass portion comprises syngas produced directly from the dry reforming of methane and that is not subjected to the cryogenic separation of carbon monoxide.

4. The method of claim 3, wherein the bypass portion comprises a volume percent of the syngas discharged from the dry reforming process in a range of 5% to 40%.

5. The method of claim 1, wherein the hydrogenation of carbon monoxide comprises a reaction:
2H₂+CO→CH₃OH.

6. The method of claim 1, wherein the synthesizing of ethanol via methanol homologation comprises reaction:
CH₃OH+H₂+CO→C₂H₅OH.

7. The method of claim 1, wherein generating the ethanol from the methanol, hydrogen, and the first stream containing carbon monoxide by the homologation of methanol comprises a reaction:
H₂+CO→CH₃OH—C₂H₅OH.

8. A method of producing ethanol, comprising:
converting methane and carbon dioxide in a dry reformer vessel into hydrogen (H₂) and carbon monoxide (CO);
discharging a syngas stream from the dry reformer vessel via a dry-reformer discharge conduit to a cryogenic separator comprising a cryogenic distillation column, wherein the syngas comprises hydrogen, carbon monoxide, and carbon dioxide;
cryogenically separating carbon monoxide from the syngas using the cryogenic separator;
discharging from the cryogenic separator a first stream comprising primarily carbon monoxide to a methanol homologation reaction vessel;
discharging from the cryogenic separator a second stream comprising hydrogen, carbon monoxide, and carbon dioxide via a feed conduit to a methanol synthesis system comprising a methanol synthesis reaction vessel;
bypassing a portion of the syngas from the dry reforming discharge conduit around the cryogenic separator to the feed conduit; and
synthesizing methanol (CH₃OH) from the second stream in the methanol synthesis system via hydrogenation of the carbon monoxide from the second stream in the methanol synthesis reaction vessel.

9. The method of claim 8, comprising:
discharging methanol from the methanol synthesis system to the methanol homologation system comprising a reactor vessel; and
bypassing a stream of hydrogen from a hydrogen source including steam methane reforming process (SMR), electrolysis, renewable electrolysis into the methanol homologation system.

10. The method of claim 9, wherein a catalyst of transition metals including rutheium, cobalt, nickel, iron is selected for the methanol homologation process.

11. The method of claim 9, wherein the first stream from the cryogenic separator comprising primarily of carbon monoxide is fed via a feed conduit into the methanol homologation reactor vessel.

12. The method of claim 9, wherein generating the ethanol from methanol undergoes homologation comprises a reaction:
CH₃OH+CO+2H₂→C₂H₅OH+H₂O.

13. The method of claim 8, wherein the hydrogenation of the carbon monoxide comprises a reaction:
2H₂+CO→CH₃OH.

14. A system for producing ethanol comprising:
a dry reformer vessel to convert methane (CH₄) and carbon dioxide (CO₂) into hydrogen (H₂) and carbon monoxide (CO) and discharge synthesis gas (syngas) comprising hydrogen, carbon monoxide, and carbon dioxide;
a cryogenic separator to receive the syngas discharged from the dry reformer vessel and cryogenically separating carbon monoxide from the syngas to discharge a first stream comprising primarily carbon monoxide to a methanol homologation system operationally coupled to the cryogenic separator and discharge a second stream comprising hydrogen, carbon monoxide, and carbon dioxide to a methanol synthesis system comprising a methanol synthesis reactor vessel, wherein the cryogenic separator comprises a cryogenic distillation column, and wherein the methanol homologation system comprises a methanol homologation reactor vessel;
a bypass conduit to discharge a portion of the syngas formed in the dry reformer vessel around the cryogenic separator to the second stream; and
the methanol synthesis system to produce methanol (CH₃OH) from the second stream by hydrogenation of the carbon monoxide from the second stream in the methanol synthesis reactor vessel.

15. The system of claim 14, wherein the dry reformer vessel converts the methane and carbon dioxide to hydrogen and carbon monoxide comprises a reaction:
CO₂+CH₄→2H₂+2CO.

16. The system of claim 14, comprising a flow control valve placed along the bypass conduit to control an amount of the syngas discharged from the dry reformer vessel that bypasses around the cryogenic separator to the second stream.

17. The system of claim 14, comprising a methanol homologation reactor vessel operationally coupled to the methanol synthesis system to receive methanol discharged from the methanol synthesis system to undergo homologation reaction with hydrogen, carbon monoxide, and methanol.

18. The system of claim 17, wherein the methanol homologation reaction forms ethanol by the reaction of the first stream primarily comprising of carbon monoxide, hydrogen, and methanol by a reaction:
CH₃OH+CO+2H₂→C₂H₅OH+H₂O.

19. The system of claim 14, wherein the hydrogenation of the carbon monoxide comprises a reaction:
2H₂+CO→CH₃OH.
